(19) United States
(12) Patent Application Publication
(10) Pub. No.: US 2026/0262970 A1
(43) Pub. Date: Sep. 10, 2026

(54) CONVERSION METHOD FOR MEASUREMENT VALUE OF SALIVA, INFORMATION PROCESSING DEVICE, AND PROGRAM

(71) Applicant: ARKRAY, Inc., Kyoto-shi (JP)

(72) Inventors: Isao FUKUTA, Kyoto-Shi (JP); Norio INAMURA, Kyoto-shi (JP); Kizuki ICHIMI, Kyoto-shi (JP)

(73) Assignee: ARKRAY, Inc., Kyoto-shi (JP)

(21) Appl. No.: 19/658,382

(22) Filed: Apr. 24, 2026

Related U.S. Application Data
(63) Continuation of application No. PCT/JP2024/038231, filed on Oct. 25, 2024.

(30) Foreign Application Priority Data
Oct. 25, 2023 (JP) 2023-183551

(51) Int. Cl. A61B 5/145; A61B 5/00; A61B 5/1455

(52) U.S. Cl. CPC A61B 5/14546; A61B 5/1455; A61B 5/7271; A61B 2562/0295

(57) ABSTRACT

A method for converting a measurement value related to saliva includes: acquiring, from a measurement value of a predetermined measurement item using one of an expectorated mouth-rinse liquid and stimulated saliva collected from a subject, a converted value in a case of using another of the expectorated mouth-rinse liquid and the stimulated saliva collected from the subject, based on a correlation obtained in advance between an index measurement value of the predetermined measurement item measured in advance using the expectorated mouth-rinse liquid and an index measurement value of the predetermined measurement item measured in advance using the stimulated saliva, wherein at least one of cariogenic bacteria, occult blood, and leukocytes is selected as the predetermined measurement item.

### CROSS-REFERENCE TO RELATED APPLICATION

This application is a continuation application of International Application PCT/JP2024/038231 filed on Oct. 25, 2024 and designated the U.S., and this application is based upon and claims the benefit of priority of the prior Japanese Patent Application No. 2023-183551, filed on Oct. 25, 2023, the entire contents of which are incorporated herein by reference.

### FIELD

The present invention relates to a method for converting a measurement value of saliva, an information processing device, and a program.

### BACKGROUND

It is important to know the risk of oral diseases or the level of oral diseases for the prevention or treatment of oral diseases. The risk of oral diseases refers to, for example, caries risk, that is, the degree of dental caries susceptibility of the oral cavity, and periodontal disease risk, that is, the degree of susceptibility of the oral cavity to periodontal disease. The level of oral disease refers to, for example, the degree of the caries risk and the level of periodontal disease. An inspection of the risk of oral disease or the level of oral disease is performed by using saliva collected from a subject as a test sample and measuring individual components or properties that reflect the risk of oral diseases or the level of oral disease in the test sample.

As a method for collecting saliva, a method, in which saliva (referred to as resting saliva or unstimulated saliva) accumulated in the oral cavity for a certain period of time and expectorated is collected, is often used. In addition, in order to easily collect a large amount of saliva in a short time, a method for collecting saliva (referred to as stimulated saliva), which is secreted by chewing gum or the like and expectorated, is also often used. Meanwhile, in recent years, there is a method for collecting a liquid (referred to as expectorated mouth-rinse liquid) expectorated after taking a mouth-rinse liquid in the mouth and rinsing the mouth, so as to collect saliva more simply and to cope with subjects who cannot chew gum or subjects who have difficulty secreting saliva; and devices for measuring individual components or properties using the expectorated mouth-rinse liquid are also known.

### Citation List

### Patent Literature

[PTL 1] WO 2017/175673
[PTL 2] WO 2012/090995

### SUMMARY

In the case of performing an inspection to know the risk of oral disease or the level of oral disease, any of the above-described methods for collecting saliva has been performed. However, in a case where various methods are used to collect saliva, the amount of components (measurement items) or a ratio between the components in the test samples may vary even if the saliva is collected from the same subject. Therefore, even for the same measurement item, different measurement results (measurement values) may be obtained. Therefore, in saliva inspection, in a case where the measurement item is measured over a plurality of times for the same subject it has been recommended to rely on only a single method for collecting saliva, so that comparison depending on the past measurement results is possible. However, depending on a saliva collection environment, it is sometimes difficult to collect saliva by the same collection method every time. For example, there is a case where neither gum for obtaining the stimulated saliva nor mouth-rinse liquid for obtaining the expectorated mouth-rinse liquid is available, or there is a case where there is no time to allow saliva to be accumulated in the oral cavity for a certain period of time.

An object of the present disclosure is to provide: a conversion method capable of converting a measurement value obtained using one of an expectorated mouth-rinse liquid and stimulated saliva into a converted value in a case where the other of the expectorated mouth-rinse liquid and the stimulated saliva is used for a predetermined measurement item; an information processing device (apparatus); and a program.

As a result of intensive efforts, the inventors of the present application have found that there is a high correlation between a measurement value obtained using an expectorated mouth-rinse liquid and a measurement value obtained using stimulated saliva for a predetermined measurement item, and have completed the following invention.

[1] A method for converting a measurement value related to saliva, the method including acquiring, from a measurement value of a measurement item using one of an expectorated mouth-rinse liquid and stimulated saliva collected from a subject, a converted value in a case of using the other of the expectorated mouth-rinse liquid and the stimulated saliva collected from the subject, based on a correlation obtained in advance between an index measurement value of the measurement item measured using the expectorated mouth-rinse liquid and an index measurement value of the measurement item measured in advance using the stimulated saliva,
in which at least one of cariogenic bacteria, occult blood, and leukocytes is selected as the measurement item.

[2] The method according to [1], in which the measurement item is the leukocytes.

[3] The method according to [1], in which the measurement value is data indicating optical information of a test pad which has a reaction system corresponding to the measurement item and on which one of the expectorated mouth-rinse liquid and the stimulated saliva is spotted.

[4] An information processing device including: a controller that acquires, from a measurement value of a measurement item using one of an expectorated mouth-rinse liquid and stimulated saliva collected from a subject, a converted value in a case of using the other of the expectorated mouth-rinse liquid and the stimulated saliva collected from the subject, based on a correlation obtained in advance between an index measurement value of the measurement item measured using the expectorated mouth-rinse liquid and an index measurement value of the measurement item measured in advance using the stimulated saliva, and outputs the converted value, in which at least one of cariogenic bacteria, occult blood, and leukocytesis selected as the measurement item.

[5] The information processing device according to [4], in which the measurement value is data indicating optical information of a test pad which has a reaction system corresponding to the measurement item and on which one of the expectorated mouth-rinse liquid and the stimulated saliva is spotted.

[6] The information processing device according to [4] or [5], in which the controller acquires the converted value by executing a calculation procedure for obtaining the converted value from the measurement value, based on the correlation.

[7] The information processing device according to [4] or [5] , further including a storage unit that stores information indicating the correlation, in which the controller acquires the information indicating the correlation by reading the information indicating the correlation from the storage unit.

[8] The information processing device according to [4] or [5], further including a communication interface that receives information from an external device, in which the controller acquires information indicating the correlation by receiving the information indicating the correlation from the external device through the communication interface.

[9] A program for causing a computer to execute: acquiring, from a measurement value of a measurement item using one of an expectorated mouth-rinse liquid and stimulated saliva collected from a subject, a converted value in a case of using the other of the expectorated mouth-rinse liquid and the stimulated saliva collected from the subject, based on a correlation between an index measurement value of the measurement item measured in advance using the expectorated mouth-rinse liquid and an index measurement value of the measurement item measured in advance using the stimulated saliva, and outputting the converted value, in which at least one of cariogenic bacteria, occult blood, and leukocytes is selected as the measurement item.

Note that the program is a computer program, and a non-transitory computer-readable storage medium in which the program is recorded can also be included in the present invention.

According to the present invention, it is possible to convert a measurement value obtained using one of an expectorated mouth-rinse liquid and stimulated saliva into a converted value in a case where the other of the expectorated mouth-rinse liquid and the stimulated saliva is used for a measurement item. As a result, it is possible to estimate, from saliva collected using one collection method, the measurement value of saliva collected using the other collection method.

### BRIEF DESCRIPTION OF DRAWINGS

FIG. 1A shows one strip-shaped test paper to which test pads for respective measurement items are attached.

FIG. 1B shows components involved in reaction systems, which are contained in the test pads for the respective measurement items.

FIG. 2 shows a configuration example of an inspection device.

FIG. 3 is a graph and a table showing a correlation between specimens when the measurement item is buffering capacity.

FIG. 4 is a graph and a table showing a correlation between specimens when the measurement item is acidity.

FIG. 5 is a graph and a table showing a correlation between specimens when the measurement item is cariogenic bacteria.

FIG. 6 is a graph and a table showing a correlation between specimens when the measurement item is occult blood.

FIG. 7 is a graph and a table showing a correlation between specimens when the measurement item is protein.

FIG. 8 is a graph and a table showing a correlation between specimens when the measurement item is leukocytes.

FIG. 9 is a graph and a table showing a correlation between specimens when the measurement item is ammonia.

FIG. 10 is a flowchart showing a processing example of an information processing device.

### DESCRIPTION OF EMBODIMENTS

### Saliva Collection Method

Saliva as a test sample (also referred to as a specimen) collected from an oral cavity includes resting saliva, stimulated saliva, and an expectorated mouth-rinse liquid. The resting saliva is collected from a subject expectorating saliva accumulated in the oral cavity for a certain period of time (for example, 5 minutes). The stimulated saliva is collected, for example, from a subject chewing gum for saliva collection for 5 minutes and expectorating saliva accumulated in the oral cavity. An expectorated mouth-rinse liquid is collected, for example, from a subject taking purified water into the mouth and expectorating the purified water. For example, the expectorated mouth-rinse liquid is collected from a subject taking 3 mL of purified water (mouth-rinse water) into the mouth and expectorating the mouth-rinse liquid obtained by rinsing in the oral cavity for 10 seconds. A volume of the purified water and a time for which the purified water is contained in the mouth can be appropriately changed as necessary. The obtained test sample can be used for subsequent operations without particular pretreatment, but additional operations such as dilution may be appropriately performed as necessary. In the present application, the expectorated mouth-rinse liquid and the resting saliva are employed as the test samples.

### Measurement Item

Measurement items related to saliva include buffering capacity, acidity, cariogenic bacteria, occult blood, protein, leukocytes, and ammonia. Details of formulation of indicators (measurement principles) for measuring measurement values of the measurement items are as follows as an example. In the present application, at least one selected from among the cariogenic bacteria, the occult blood, and the leukocytes among the seven measurement items is employed as a predetermined measurement item (measurement target).

### Buffering Capacity

The measurement value of the buffering capacity of saliva is preferably measured using, for example, a pH indicator. As the measurement principle, a principle in which, when bringing the test sample into contact with an absorbent carrier containing an acidic buffer and the pH indicator in advance, an indicated pH of the indicator approaches an original pH of saliva as acid buffering capacity is higher, and approaches an acidic range lower than the original pH of saliva as the acid buffering capacity is lower, is utilized. The pH indicator may be a mixture of a plurality of reagents. For example, a combined reagent of bromocresol green and bromoxylenol blue can be used. As the acidic buffer, for example, a nonvolatile organic acid can be used. Examples of the nonvolatile organic acid include citric acid, malic acid, tartaric acid, malonic acid, oxalic acid, sulfosalicylic acid, sulfanilic acid, benzoic acid, and tricarballylic acid. Among the acids, tartaric acid is more preferable. As the acidic buffer, an inorganic acid such as metaphosphoric acid can also be used. The acidic buffer may be, for example, a buffer such as a mixture of potassium hydrogen phthalate and potassium phosphate. A concentration of the reagent can be appropriately set, and a reaction time can be appropriately set. In addition, a detection condition when the buffering capacity is detected by a detection device such as a measurement device 20 described below can be appropriately set.

### Acidity

The measurement value of the acidity of saliva is preferably measured using, for example, a pH indicator. As the pH indicator, any known pH indicator can be used, and it is preferable to use a pH indicator having a color change range from pH 2 to 9, and it is more preferable to use a pH indicator having a color change range from pH 3 to 8. For example, sodium bromothymol blue, or a combined reagent of bromocresol green and bromoxylenol blue can be suitably used. A concentration of the pH indicator can be appropriately set, and a reaction time can be appropriately set. In addition, a detection condition when performing detection using a detection device such as the measurement device 20 described below can be appropriately set.

### Cariogenic Bacteria

The measurement value of the cariogenic bacteria (Mutans streptococci) is, for example, a count of the cariogenic bacteria. The cariogenic bacteria can be measured, for example, by a method using a reduction reaction of resazurin or a method using an antibody against Mutans streptococci. The method using the reduction reaction of resazurin is referred to as a resazurin method. Resazurin is an oxidation-reduction indicator and usually exists as resazurin (maximum absorption wavelength: 605 nm), which is an oxidized blue dye. However, resazurin is reduced by NADH produced by metabolism of Gram-positive bacteria including Mutans streptococci and converted into resorufin, which is a reddish-purple dye (maximum absorption wavelength: 573 nm). That is, the reduction of resazurin proceeds according to a count of viable Mutans streptococci. In a case where the resazurin method is used, a measurement reagent preferably further contains 1-methoxy-5-methylphenazinium methyl sulfate (methoxy-PMS) in addition to resazurin. In a case where methoxy-PMS is contained, it is effective to perform measurement at room temperature under a short-time reaction condition as compared with a case where methoxy-PMS is not contained. A concentration of the reagent can be appropriately set, and a reaction time can be appropriately set. In addition, a detection condition when performing detection using a detection device such as the measurement device 20 described below can be appropriately set.

### Occult Blood

The occult blood is blood in the test sample obtained from the oral cavity. The measurement value of the occult blood is, for example, an amount or a concentration of blood in the test sample obtained from the oral cavity. It is known that the occult blood is detected in saliva when gingival tissue is destroyed due to periodontal disease, and the occult blood is an item reflecting the degree of destruction of periodontal tissue. As an amount of the occult blood (occult blood amount) increases, the degree of destruction of the periodontal tissue tends to be higher, and a periodontal disease stage tends to be more severe. A method for measuring the measurement value of the occult blood is not particularly limited, and any known method can be used. For example, the measurement is preferably performed by a hemoglobin catalytic activity method. The hemoglobin catalytic activity method uses an ability (peroxidase-like activity) of hemoglobin, myoglobin, or degradation products thereof, which are blood components, to catalyze transfer of oxygen from an oxygen donor such as a peroxide to an oxygen acceptor. By using an indicator whose color tone changes due to oxidation as the oxygen receptor, the occult blood is measured through measurement of a concentration of hemoglobin or the like by measuring a color reaction. A concentration of the reagent can be appropriately set, and a reaction time can be appropriately set. In addition, a detection condition when performing detection using a detection device such as the measurement device 20 described below can be appropriately set.

### Protein

The protein is a protein in the test sample obtained from the oral cavity. The measurement value of the protein is, for example, the total concentration of the protein in the test sample obtained from the oral cavity. Since the total concentration of the protein increases as a count of periodontal bacteria in the oral cavity increases, the total concentration of the protein is an item reflecting the count of the periodontal bacteria. As the total concentration of the protein increases, the periodontal disease stage tends to be more severe. A method for measuring the measurement value of the protein is not particularly limited, and any known method can be used. For example, the measurement is preferably performed by a protein error method. The protein error method is a method using a phenomenon in which a pH indicator indicates a pH higher than a true pH of a solution in proportion to the concentration of the protein, and the protein can be quantified from color development of the indicator. A concentration of the reagent can be appropriately set, and a reaction time can be appropriately set. In addition, a detection condition when performing detection using a detection device such as the measurement device 20 described below can be appropriately set.

### Leukocytes

The leukocytes are leukocytes in the test sample obtained from the oral cavity. The measurement value of the leukocytes is, for example, a count of the leukocytes (leukocyte count) in the test sample obtained from the oral cavity. It is known that the leukocytes gather at a site (inflammatory site) affected by the periodontal disease, and the leukocyte count is an item reflecting the degree of inflammation of the periodontal tissue. As the leukocyte count increases, the degree of inflammation of the periodontal tissue tends to be higher, and the periodontal disease stage tends to be more severe. A method for measuring the measurement value of the leukocytes is not particularly limited, and any known method can be used. For example, the measurement is preferably performed by a leukocyte esterase method. The leukocyte esterase method uses the fact that the leukocytes increase when tissue becomes inflamed, and production of esterase by the leukocytes also increases accordingly. This is a technique for measuring esterase activity by directly causing color development of an alcohol (phenol) component produced using an ester compound as a substrate, which is hydrolyzed by esterase (leukocyte esterase) produced by the leukocytes or by causing color development through coupling of the alcohol (phenol) with a diazonium salt. Leukocytes can be calculated based on the measurement value of esterase activity. A concentration of the reagent can be appropriately set, and a reaction time can be appropriately set. In addition, a detection condition when performing detection using a detection device such as the measurement device 20 described below can be appropriately set.

### Ammonia

The ammonia is ammonia in the test sample obtained from the oral cavity. The measurement value of the ammonia is, for example, a concentration of the ammonia (ammonia concentration) in the test sample obtained from the oral cavity. The ammonia is an item reflecting a count of bacteria in the oral cavity since the concentration of the ammonia increases as bacteria including the periodontal bacteria actively propagate in the oral cavity. As the concentration of the ammonia increases, the periodontal disease stage tends to be more severe. A method for measuring the measurement value of the ammonia is not particularly limited, and any known method can be used. For example, it is preferable to perform the measurement by a microdiffusion method (Conway's method). The microdiffusion method is a method used for quantification of ammonia nitrogen, and is a method in which ammonia gas volatilized from the test sample, which has been made alkaline with an alkaline buffer, is trapped in an absorbing solution or the like, and color development of an indicator is quantified by a method such as colorimetry. As another method, an ammonia quantification method disclosed in Japanese Patent No. 7328739 can be applied. The ammonia quantification method is a method for quantifying the ammonia in an ammonia-containing sample such as saliva by performing a glutamic acid synthetase reaction and quantifying the ammonia in the ammonia-containing sample based on an amount of the ammonia consumed in the reaction, and a chelating agent is present in a glutamine synthetase reaction system. Any type of chelating agent may be used as long as the chelating agent can exert a chelating effect on calcium ions. The glutamine synthetase reaction consumes the ammonia and produces the same amount (equimolar) of ADP and orthophosphate as the ammonia. The ammonia can be quantified by measuring the amount of the ADP or phosphate produced or an amount of a product of a reaction using the ADP or phosphate. An ammonia quantification reagent used in the quantification method may be any reagent as long as the reagent contains the chelating agent, glutamine synthetase, ATP, and glutamic acid described above. In a case where the ammonia is quantified based on the ADP or phosphate produced by the glutamine synthetase reaction, the ammonia quantification reagent preferably contains a reagent for measuring the amount of the ADP or phosphate. A concentration of the reagent can be appropriately set, and a reaction time can be appropriately set. In addition, a detection condition when performing detection using a detection device such as the measurement device 20 described below can be appropriately set.

### Correlation

In the present application, for the above-described measurement items, it has been found that a measurement value obtained using one of the expectorated mouth-rinse liquid and the stimulated saliva has a high correlation with a measurement value in a case where the other of the expectorated mouth-rinse liquid and the stimulated saliva collected from the same subject is used. In the present invention, from a measurement value of the predetermined measurement item measured using one of the expectorated mouth-rinse liquid and the stimulated saliva collected from the subject, a converted value in a case where the other of the expectorated mouth-rinse liquid and the stimulated saliva collected from the subject is used is acquired (that is, the measurement value is converted into the converted value) based on the correlation.

Here, the correlation is a relationship in which two matters or values are associated with each other, and indicates a tendency that when one increases or decreases, the other also increases or decreases. The correlation can be shown using, for example, a simple regression equation (y=ax+b) based on simple regression analysis using a measurement value in the case of using the expectorated mouth-rinse liquid and a measurement value obtained using the stimulated saliva. In the simple regression equation, x is an explanatory variable, y is an objective variable, a is a regression coefficient, and b is an intercept. The explanatory variable x is a measurement value for one of the expectorated mouth-rinse liquid and the stimulated saliva, and the objective variable y represents a measurement value for the other of the expectorated mouth-rinse liquid and the stimulated saliva.

In the present specification, the measurement value for each of the expectorated mouth-rinse liquid and the stimulated saliva, which is obtained for deriving (creating) the correlation shown by the simple regression equation or the like described above, is referred to as an index measurement value. In other words, the measurement value for the expectorated mouth-rinse liquid or the stimulated saliva, which is obtained to calculate a regression line in the simple regression equation, corresponds to the index measurement value. For example, the simple regression equation described above is used as a conversion formula. The measurement value for one of the expectorated mouth-rinse liquid and the stimulated saliva collected from the subject is substituted into the simple regression equation as the explanatory variable x. The objective variable y is calculated by substitution, and the calculated objective variable y is treated as the converted value for the other of the expectorated mouth-rinse liquid and the stimulated saliva.

The number of measurements for the index measurement value for obtaining the correlation can be an arbitrary number. The number of measurements for the expectorated mouth-rinse liquid for acquiring the index measurement value and the number of measurements for the stimulated saliva may be the same as each other or different from each other. As long as an increase or decrease in one indicates an increase or decrease in the other, the correlation may be shown using something other than the simple regression equation.

The index measurement value of the measurement item may be a value indicating primary data used for calculating the measurement value of the predetermined measurement item. The primary data may be, for example, data indicating optical information such as reflectance and transmittance of a test pad which has a reaction system corresponding to the predetermined measurement item and on which the expectorated mouth-rinse liquid or the stimulated saliva is spotted. In addition, the index measurement value may be a value indicating a quantity of the predetermined measurement item obtained from the primary data using a calibration curve or an arithmetic expression. In other words, the index measurement value may be information directly indicating a quantity representing the measurement item or may be information indirectly indicating the quantity representing the measurement item, such as reflectance. A specimen provider for obtaining the index measurement value and the subject from which the measurement value is obtained may be the same person or different persons.

The measurement value of the measurement item related to the expectorated mouth-rinse liquid or the stimulated saliva collected from the subject may be a value indicating primary data used for calculating the measurement value of the predetermined measurement item, similarly to the index measurement value. The primary data may be, for example, data indicating optical information such as reflectance and transmittance of a test pad which has a reaction system corresponding to the predetermined measurement item and on which the expectorated mouth-rinse liquid or the stimulated saliva is spotted. In addition, the measurement value may be a value indicating the quantity of the predetermined measurement item obtained from the primary data using a calibration curve or an arithmetic expression. In other words, the measurement value may be information directly indicating a quantity representing the measurement item or may be information indirectly indicating the quantity representing the measurement item, such as reflectance.

Hereinafter, a method for converting a measurement value related to saliva and an information processing device used therefor according to the present embodiment will be described with reference to the drawings. The examples described below are merely exemplary, and the present invention is not limited to the examples.

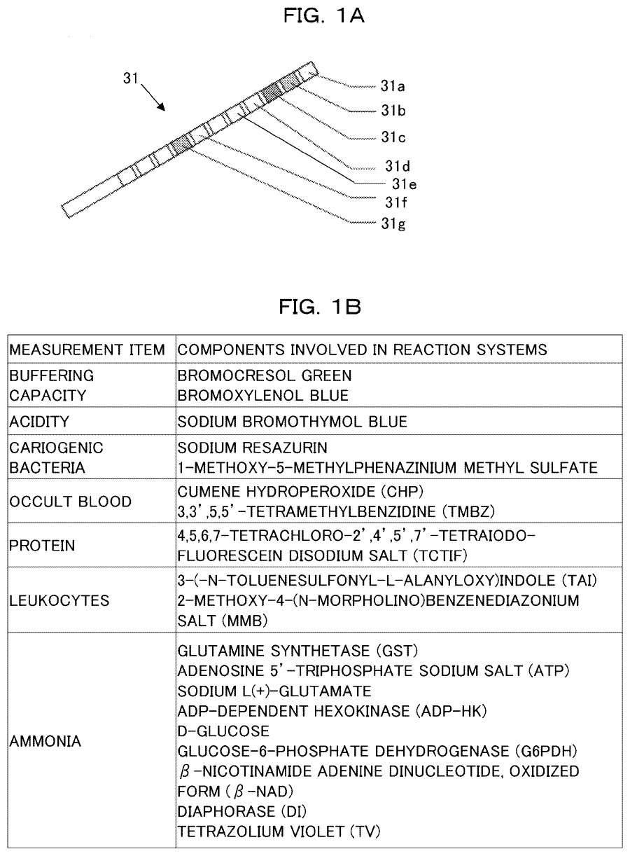

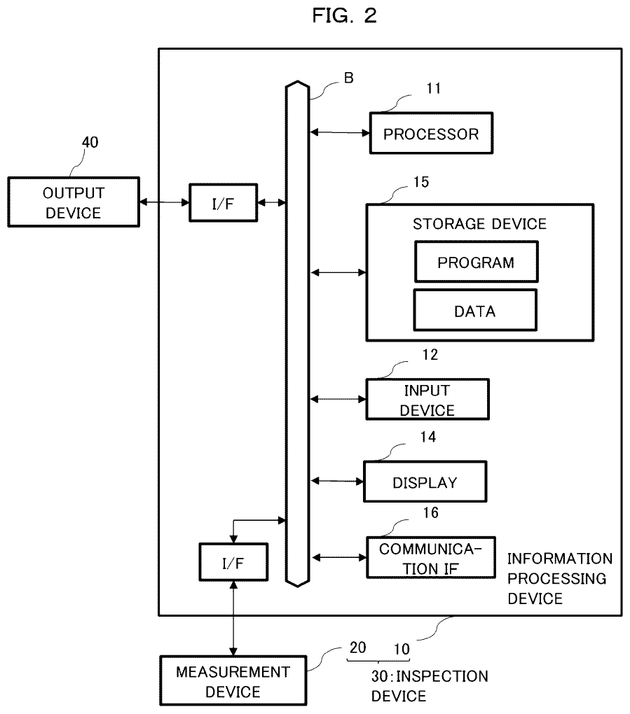

FIG. 1A shows an example of one strip-shaped test paper 31 to which test pads 31a to 31g impregnated with the indicators (components involved in the reaction systems) for measuring the seven measurement items described above are attached. The test pad 31a is used to measure the buffering capacity. The test pad 31b is used to measure the acidity. The test pad 31c is used to measure the cariogenic bacteria. The test pad 31d is used to measure the occult blood. The test pad 31e is used to measure the protein. The test pad 31e is used to measure the leukocytes. The test pad 31f is used to measure the ammonia. FIG. 1B shows components involved in the reaction systems, which are contained in the test pads 31a to 31g for the respective measurement items. When the test sample is spotted to the test pads 31a to 31g of the strip-shaped test paper 31, the color tone changes according to the reaction principle according to each measurement item described above. The color tone is measured by the measurement device 20 (FIG. 2).

FIG. 2 shows a configuration example of an inspection device 30. The inspection device 30 includes an information processing device 10 and the measurement device 20. The information processing device 10 is a dedicated or general-purpose computer such as a personal computer (PC), a workstation, or a smart device (smartphone or tablet terminal). The information processing device 10 includes a processor 11 (corresponding to a control unit and a controller), a storage device 15 (corresponding to a storage unit), an input device 12, a display (display device) 14, and a communication interface (communication IF) 16, which are connected to a bus B. The measurement device 20 and an output device 40 are electrically connected to the bus B of the information processing device 10 via an interface circuit (I/F).

The measurement device 20 measures, as the predetermined measurement item in the test sample obtained from the oral cavity of the subject, the measurement values of the buffering capacity, the acidity, the cariogenic bacteria, the occult blood, the protein, the leukocytes, and the ammonia. The measurement device 20 measures the color tones of the test pads 31a to 31g to which the test sample is spotted by reflectance photometry, and calculates (detects) the reflectance. The measurement value of each measurement item is obtained from the reflectance. As the measurement device 20, for example, a measurement device “SillHa” capable of measuring six items excluding the occult blood among the seven measurement items or a measurement device “ST-4910” (both manufactured by ARKRAY, Inc.) capable of measuring the seven items can be used. In addition, it is possible to use the strip-shaped test paper 31 capable of measuring each measurement item, which is compatible with the measurement devices. The measurement device 20 can be implemented by one or two or more measurement devices capable of performing measurement for one or two or more measurement items. In addition, the number of strip-shaped test papers and the number of test pads included in each strip-shaped test paper are arbitrary as long as the measurement result for the predetermined measurement item can be obtained. However, the reflectance photometry is only an example of a method for measuring each measurement item, and different measurement methods may be used, and the measurement device 20 is not limited to the above-described configuration. In addition, it is sufficient if the measurement method is appropriately set according to the formulation (measurement principle) of the indicator for the measurement item.

The storage device 15 includes a main storage device and an auxiliary storage device. The storage device 15 stores a program to be executed by the processor 11 and data used for executing the program. The storage device 15 includes a read only memory (ROM), a random access memory (RAM), a hard disk drive, a solid state drive (SSD), and the like. The storage device 15 may be installed (fixed arrangement) in the information processing device 10, or may be detachable (universal serial bus (USB) memory or the like) from the information processing device 10 via a USB connector or the like. As described below, the storage device 15 stores information indicating the correlation, and for example, in a case where the information indicating the correlation is the simple regression equation, information indicating the simple regression equation can also be stored as data. The information indicating the correlation can be stored in the form of a program for calculating the objective variable y (converted value) by substituting the explanatory variable x (measurement value), or in the form of reading the corresponding objective variable y from a correspondence table according to an input of the explanatory variable x. Alternatively, the information indicating the correlation can be stored in the form of a program that inquires of a communication partner (such as a server connected to the information processing device 10 via a network) the objective variable y corresponding to the input explanatory variable x and receives the corresponding objective variable y as an answer (response) to the inquiry.

The input device 12 is a key, a button, a touch panel, or the like, and is used to input data. For the measurement value of the measurement device 20, a signal of data indicating the measurement result may be input (received) from the measurement device 20 to the information processing device 10 via an interface, or may be manually input using the input device 12. The display 14 displays data and information. The communication IF 16 can also be used to communicate with an external device or the like and acquire the information indicating the correlation through communication with the external device. The data indicating the measurement value may be obtained by wireless communication with the measurement device 20. The communication IF 16 may be installed (fixed arrangement) in the information processing device 10 or may be detachable via a USB connector or the like.

The processor 11, which is a controller, is a central processing unit (CPU) or the like, and performs various types of processing by executing the program stored in the storage device 15. For example, the processor 11 can convert the measurement value based on the correlation described above into the converted value by executing the program. That is, it is possible to perform processing of converting the measurement value in the case of using the expectorated mouth-rinse liquid into the converted value in the case of using the stimulated saliva, based on the correlation. In addition, it is possible to perform processing of converting the measurement value of the measurement item using the stimulated saliva into the converted value in the case of using the expectorated mouth-rinse liquid, based on the correlation.

As described above, the conversion from the measurement value to the converted value can be performed, for example, by substituting the measurement value that is the explanatory variable x into the simple regression equation (y=ax+b) indicating the correlation and calculating the objective variable y as the converted value. The storage device 15 can store a program including an algorithm (calculation procedure) for obtaining the converted value by using the conversion formula from the measurement value. For example, in a case where the measurement value that is the explanatory variable x input to or received by the information processing device 10 is acquired by execution of the program, the processor 11 can calculate the converted value that is the objective variable y by executing the calculation procedure for obtaining the objective variable y using the explanatory variable x and the regression coefficient a and the intercept b stored in advance in the storage device 15. In addition, the conversion from the measurement value to the converted value may be performed, for example, by storing a correspondence relationship between the measurement value (explanatory variable x) and the converted value (objective variable y) in the storage device 15 in advance using the correlation (simple regression equation) described above, and reading, by the processor 11, the converted value corresponding to the measurement value from the storage device 15 when the measurement value is acquired. The correspondence relationship can be stored using various data structures such as the correspondence table between the measurement value and the converted value or a relational database. Furthermore, the conversion from the measurement value to the converted value may employ a configuration in which, in a case where the measurement value is acquired, the processor 11 requests another device (communication partner) to provide the converted value, inquires the converted value, or the like, and receives (acquires) the converted value transmitted in response to the request or the inquiry. The output of the converted value is, for example, display on the display 14, output from an output device such as a printer, or transmission of data or a signal indicating the converted value. Furthermore, in order to perform the above-described conversion, a configuration in which the information processing device 10 acquires the information indicating the correlation by communication using the communication IF 16 and performs the above-described conversion using the acquired information indicating the correlation may be employed.

In addition, the processor 11 can determine, for example, the periodontal disease stage based on the measurement value or the converted value of the measurement item, and generate information indicating a result of determining the periodontal disease stage. The display 14 may display the generated information. Alternatively, the information may be printed by the output device 40 such as a printer. Furthermore, the information may be transmitted to the communication partner using the communication IF 16 or the like. Note that the processor 11 may be a device (digital signal processor (DSP), graphical processing unit (GPU), or the like) other than a CPU. Furthermore, the processing performed by the processor 11 may be performed by a combination of a plurality of types of processors, a field programmable gate array (FPGA), a complex programmable logic device (CPLD), or an integrated circuit such as an application specific integrated circuit (ASIC) or a system-on-a-chip (SoC).

### Example

From the oral cavity of the same subject, each of an expectorated mouth-rinse liquid, resting saliva, and stimulated saliva collected according to the above-described saliva collection method was prepared. That is, the expectorated mouth-rinse liquid was collected from the subject expectorating the mouth-rinse liquid obtained from the subject taking 3 mL of purified water into the mouth and rinsing in the oral cavity for 10 seconds, the resting saliva was collected from the subject expectorating saliva accumulated in the oral cavity for 5 minutes, and the stimulated saliva was collected from the subject chewing gum for collecting saliva for 5 minutes and expectorating the saliva accumulated in the oral cavity. The following experiment was conducted in order to confirm the presence or absence of the correlation between the measurement values of the respective measurement items (the buffering capacity, the acidity, the cariogenic bacteria, the occult blood, the protein, the leukocytes, and the ammonia) in such specimens collected by different collection methods and to obtain the conversion formula for converting the measurement value of the predetermined measurement item measured using one of the collection methods for which the correlation has been confirmed into the converted value in the case of using the other. Incidentally, the expectorated mouth-rinse liquid was an undiluted solution (that is, a liquid not subjected to dilution), and saliva obtained by diluting the undiluted solution 5 times with purified water (distilled water) was used as the resting saliva and the stimulated saliva.

The number (n) of specimens for measuring the buffering capacity was 15 (n=15), and the number of specimens for measuring the pH and the count of Mutans streptococci was 20 (n=20). In addition, the number of specimens for measuring the occult blood amount and the total concentration of the protein was 12 (n=12), the number of specimens for measuring the count of the leukocytes was 17 (n=17), and the number of specimens for measuring the concentration of the ammonia was 8 (n=8).

The buffering capacity, the acidity, the cariogenic bacteria, the occult blood, the protein, the leukocytes, and the ammonia for each specimen were measured by using the strip-shaped test paper 31 (FIG. 1) and the measurement device 20 (SillHa and ST-4910 for the occult blood). The strip-shaped test paper 31 has the test pads 31a to 31g corresponding to the respective measurement items. The test pads 31a to 31g contained components (FIG. 1B) involved in the reaction systems corresponding to the respective measurement items, and reflectance of each of the test pads 31a to 31g of the strip-shaped test paper 31 when saliva as the test sample was spotted on each of the test pads 31a to 31g was measured by the measurement device 20. For the cariogenic bacteria, the leukocytes, and the ammonia, one measurement was performed for each specimen, and for the buffering capacity, the acidity, the occult blood, and the protein, two measurements were performed for each specimen, and an average value of the measurement values was obtained. That is, in the present example, the measurement value obtained in one measurement corresponds to the index measurement value for the cariogenic bacteria, the leukocytes, and the ammonia, and each average value of the measurement values obtained in two measurements corresponds to the index measurement value of the predetermined measurement item for the buffering capacity, the acidity, the occult blood, and the protein.

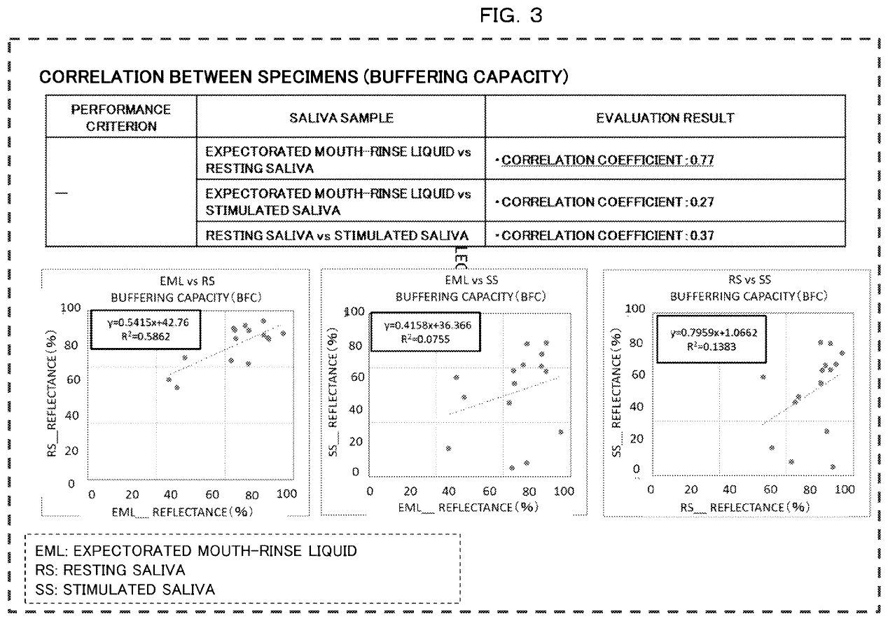

FIG. 3 is a graph and a table showing a correlation between the specimens when the measurement item is the buffering capacity. The graph on the left side of FIG. 3 is obtained by plotting the index measurement value of the buffering capacity (reflectance of the test pad 31a) for each of the expectorated mouth-rinse liquid and the resting saliva and further drawing a regression line, and shows a correlation between the index measurement value for the expectorated mouth-rinse liquid and the index measurement value for the resting saliva. A vertical axis of the graph on the left side represents the reflectance [%] of the test pad 31a on which the resting saliva is spotted, and a horizontal axis represents the reflectance [%] of the test pad 31a on which the expectorated mouth-rinse liquid is spotted. The graph at the center is obtained by plotting the index measurement value of the buffering capacity for each of the expectorated mouth-rinse liquid and the stimulated saliva, and shows a correlation between the expectorated mouth-rinse liquid and the stimulated saliva. A vertical axis of the graph at the center represents the reflectance [%] of the test pad 31a on which the stimulated saliva is spotted, and a horizontal axis represents the reflectance [%] of the test pad 31a on which the expectorated mouth-rinse liquid is spotted. The graph on the right side is obtained by plotting the index measurement value of the buffering capacity for each of the resting saliva and the stimulated saliva, and shows a correlation between the resting saliva and the stimulated saliva. A vertical axis of the graph on the right side represents the reflectance [%] of the test pad 31a on which the stimulated saliva is spotted, and a horizontal axis represents the reflectance [%] of the test pad 31a on which the resting saliva is spotted. Note that the vertical axis and the horizontal axis may be reversed, that is, the explanatory variable x and the objective variable y of the simple regression equation indicating the regression line may be reversed.

Here, it can be said that the correlation between the specimens exists when the correlation coefficient of results of plotting the index measurement values of the specimens is 0.6 or more, and the conversion can be performed. In addition, it can be said that a stronger correlation exists when the correlation coefficient is 0.7 or more, and accuracy when the conversion is performed is high, and it can be said that a much stronger correlation exists when the correlation coefficient is 0.8 or more, and accuracy when the conversion is performed is very high. Note that the correlation coefficient is not limited to being calculated based on the result of plotting the index measurement values of the respective specimens, and any means may be used as long as the means is a known method for quantifying the correlation. Although the correlation coefficient may be less than 0.6 depending on the measurement method and measurement principle, in the present embodiment, a model (y=ax+b) having a correlation coefficient of 0.6 or more is employed as the conversion formula between the specimens. The same applies to other than the buffering capacity.

The table in FIG. 3 shows the correlation coefficient between the expectorated mouth-rinse liquid and the resting saliva, the correlation coefficient between the expectorated mouth-rinse liquid and the stimulated saliva, and the correlation coefficient between the resting saliva and the stimulated saliva. The correlation coefficients were 0.77, 0.27, and 0.37, respectively. As a result, it could be determined for the buffering capacity that a correlation exists only between the expectorated mouth-rinse liquid and the resting saliva. In addition, it was found that a model y=0.7118x+0.7695 (determination function R²=0.4562) between the expectorated mouth-rinse liquid and the resting saliva can be employed as the conversion formula between the expectorated mouth-rinse liquid (x) and the resting saliva (y).

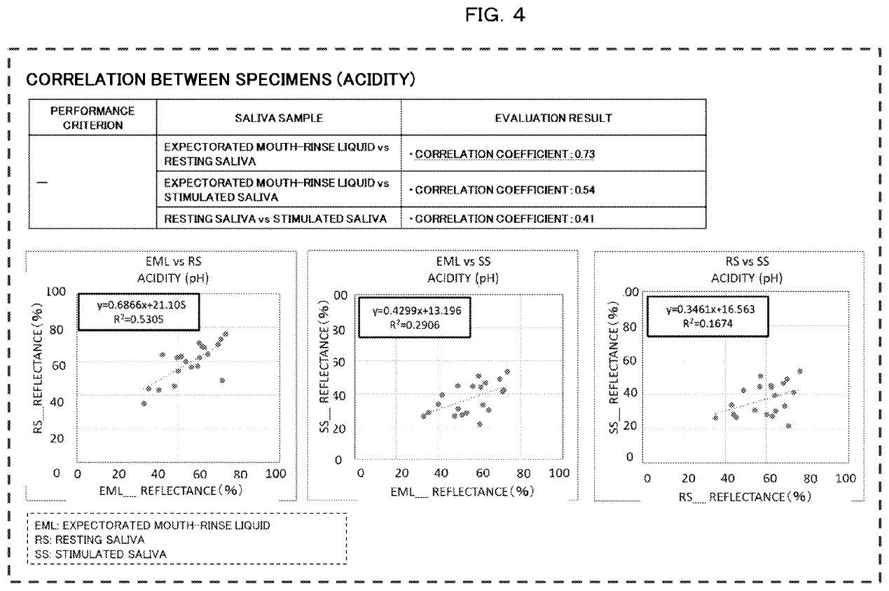

FIG. 4 is a graph and a table showing a correlation between the specimens when the measurement item is the acidity, and has the same format as the graph and the table showing the correlation between the specimens when the measurement item is the buffering capacity. Each graph shows a plot and a regression line of the index measurement value (the reflectance of the test pad 31b) of the pH, and shows a correlation of the index measurement value of each specimen.

The table in FIG. 4 shows the correlation coefficient between the expectorated mouth-rinse liquid and the resting saliva, the correlation coefficient between the expectorated mouth-rinse liquid and the stimulated saliva, and the correlation coefficient between the resting saliva and the stimulated saliva. The correlation coefficients were 0.73, 0.54, and 0.41, respectively. As a result, it could be determined for the acidity that a correlation exists only between the expectorated mouth-rinse liquid and the resting saliva. In addition, it was found that a model y=0.6866x+21.105 (determination function R²=0.5305) between the expectorated mouth-rinse liquid and the resting saliva can be employed as the conversion formula between the expectorated mouth-rinse liquid (x) and the resting saliva (y).

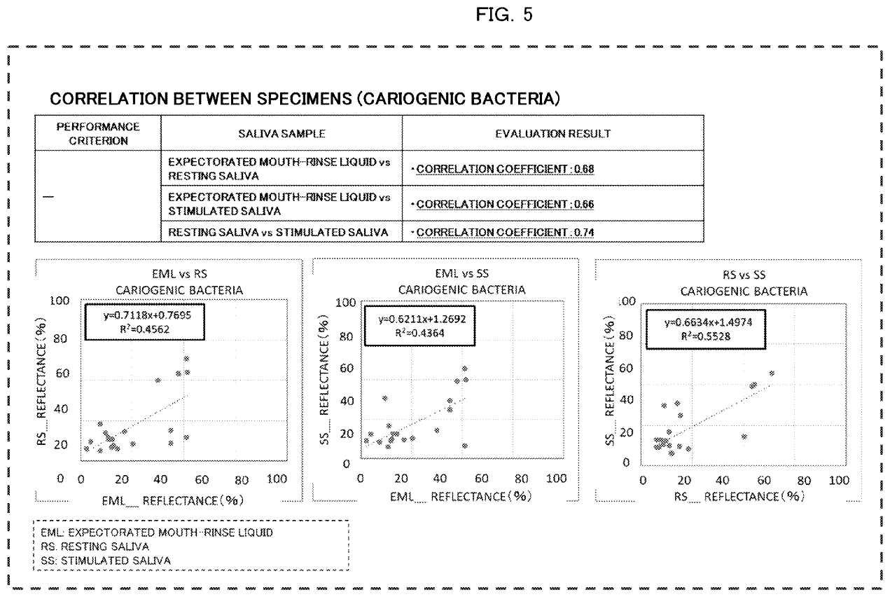

FIG. 5 is a graph and a table showing a correlation between the specimens when the measurement item is the cariogenic bacteria, and has the same format as the graph and the table showing the correlation between the specimens when the measurement item is the buffering capacity. Each graph shows a plot and a regression line of the index measurement value (the reflectance of the test pad 31c) of the cariogenic bacteria, and shows a correlation of the index measurement value of each specimen.

The table in FIG. 5 shows the correlation coefficient between the expectorated mouth-rinse liquid and the resting saliva, the correlation coefficient between the expectorated mouth-rinse liquid and the stimulated saliva, and the correlation coefficient between the resting saliva and the stimulated saliva. The correlation coefficients were 0.68, 0.66, and 0.74, respectively. As a result, it could be determined for the cariogenic bacteria that a correlation exists among all the specimens. In addition, it was found that a model y=0.7118x+0.7695 (determination function R²=0.4562) between the expectorated mouth-rinse liquid and the resting saliva can be employed as the conversion formula between the expectorated mouth-rinse liquid (x) and the resting saliva (y). In addition, it was found that a model y=0.6211x+1.2692 (determination function R²=0.4364) between the expectorated mouth-rinse liquid and the stimulated saliva can be employed as the conversion formula between the expectorated mouth-rinse liquid (x) and the stimulated saliva (y). Furthermore, it was found that a model y=0.6634x+1.4974 (determination function R²=0.5528) between the resting saliva and the stimulated saliva can be employed as the conversion formula between the resting saliva (x) and the stimulated saliva (y).

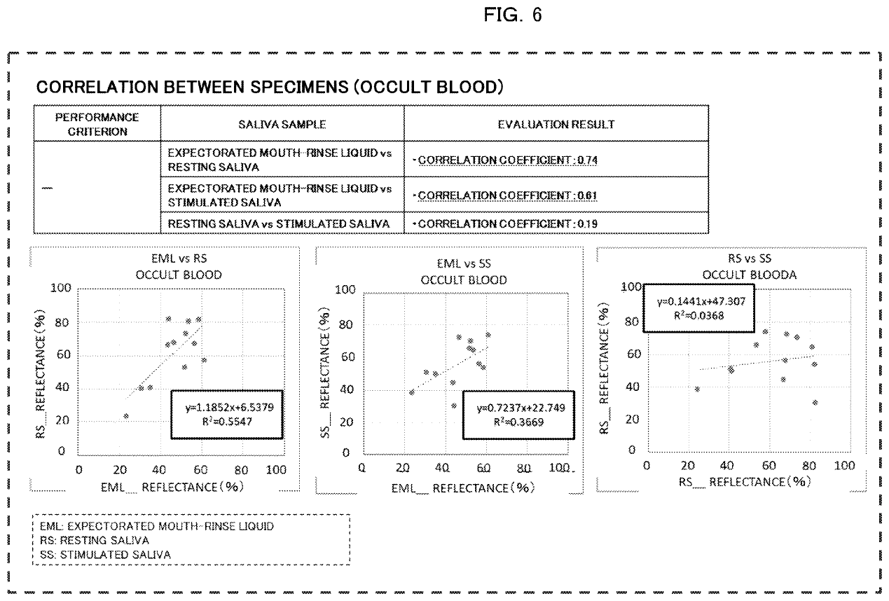

FIG. 6 is a graph and a table showing a correlation between the specimens when the measurement item is the occult blood, and has the same format as the graph and the table showing the correlation between the specimens when the measurement item is the buffering capacity. Each graph shows a plot and a regression line of the index measurement value (the reflectance of the test pad 31d) of the occult blood, and shows a correlation of the index measurement value of each specimen.

The table in FIG. 6 shows the correlation coefficient between the expectorated mouth-rinse liquid and the resting saliva, the correlation coefficient between the expectorated mouth-rinse liquid and the stimulated saliva, and the correlation coefficient between the resting saliva and the stimulated saliva. The correlation coefficients were 0.74, 0.61, and 0.19, respectively. It could be determined for the occult blood that a correlation exists between the expectorated mouth-rinse liquid and the resting saliva, and between the expectorated mouth-rinse liquid and the resting saliva. In addition, it was found that a model y=1.1852x+6.5379 (determination function R²=0.55472) between the expectorated mouth-rinse liquid and the resting saliva can be employed as the conversion formula between the expectorated mouth-rinse liquid (x) and the resting saliva (y). In addition, it was found that a model y=0.7237x+22.749 (determination function R²=0.3669) between the expectorated mouth-rinse liquid and the stimulated saliva can be employed as the conversion formula between the expectorated mouth-rinse liquid (x) and the stimulated saliva (y).

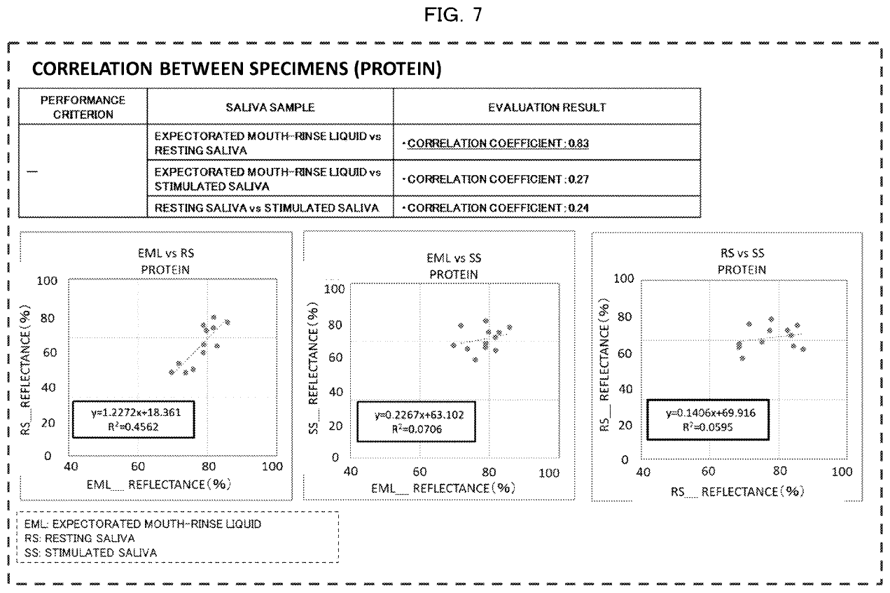

FIG. 7 is a graph and a table showing a correlation between the specimens when the measurement item is the protein, and has the same format as the graph and the table showing the correlation between the specimens when the measurement item is the buffering capacity. Each graph shows a plot and a regression line of the index measurement value (the reflectance of the test pad 31e) of the occult blood, and shows a correlation of the index measurement value of each specimen.

The table in FIG. 7 shows the correlation coefficient between the expectorated mouth-rinse liquid and the resting saliva, the correlation coefficient between the expectorated mouth-rinse liquid and the stimulated saliva, and the correlation coefficient between the resting saliva and the stimulated saliva. The correlation coefficients were 0.83, 0.27, and 0.24, respectively. It could be determined for the protein that a correlation exists only between the expectorated mouth-rinse liquid and the resting saliva. In addition, it was found that a model y=1.2272x+18.351 (determination function R²=0.5872) between the expectorated mouth-rinse liquid and the resting saliva can be employed as the conversion formula between the expectorated mouth-rinse liquid (x) and the resting saliva (y).

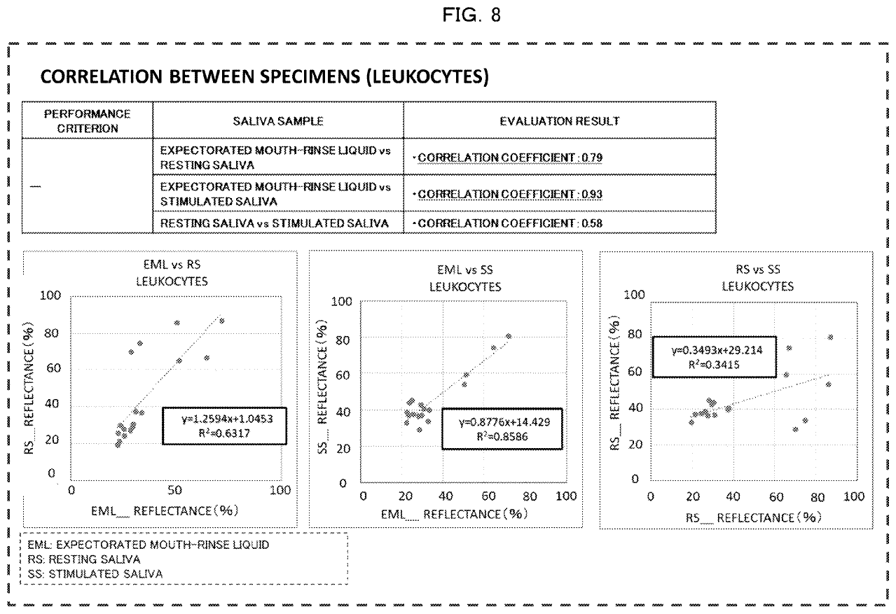

FIG. 8 is a graph and a table showing a correlation between the specimens when the measurement item is the leukocytes, and has the same format as the graph and the table showing the correlation between the specimens when the measurement item is the buffering capacity. Each graph shows a plot and a regression line of the index measurement value (the reflectance of the test pad 31f) of the leukocytes, and shows a correlation of the index measurement value of each specimen.

The table in FIG. 8 shows the correlation coefficient between the expectorated mouth-rinse liquid and the resting saliva, the correlation coefficient between the expectorated mouth-rinse liquid and the stimulated saliva, and the correlation coefficient between the resting saliva and the stimulated saliva. The correlation coefficients were 0.79, 0.93, and 0.58, respectively. It could be determined for the leukocytes that a correlation exists between the expectorated mouth-rinse liquid and the resting saliva, and between the expectorated mouth-rinse liquid and the stimulated saliva. In addition, it was found that a model y=1.2594x+1.0453 (determination function R²=0.6317) between the expectorated mouth-rinse liquid and the resting saliva can be employed as the conversion formula between the expectorated mouth-rinse liquid (x) and the resting saliva (y). In addition, it was found that a model y=0.8776x+14.429 (determination function R²=0.8586) between the expectorated mouth-rinse liquid and the stimulated saliva can be employed as the conversion formula between the expectorated mouth-rinse liquid (x) and the stimulated saliva (y).

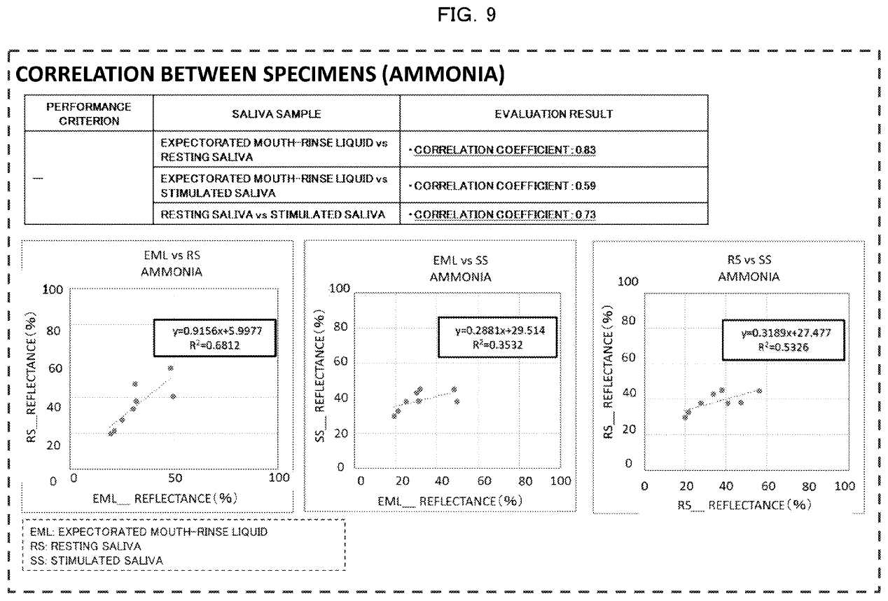

FIG. 9 is a graph and a table showing a correlation between the specimens when the measurement item is the ammonia, and has the same format as the graph and the table showing the correlation between the specimens when the measurement item is the buffering capacity. Each graph shows a plot and a regression line of the index measurement value (the reflectance of the test pad 31g) of the ammonia, and shows a correlation of the index measurement value of each specimen.

The table in FIG. 9 shows the correlation coefficient between the expectorated mouth-rinse liquid and the resting saliva, the correlation coefficient between the expectorated mouth-rinse liquid and the stimulated saliva, and the correlation coefficient between the resting saliva and the stimulated saliva. The correlation coefficients were 0.83, 0.59, and 0.73, respectively. It could be determined for the ammonia that a correlation exists between the expectorated mouth-rinse liquid and the resting saliva, and between the expectorated mouth-rinse liquid and the resting saliva. In addition, it was found that a model y=0.9156x+5.9977 (determination function R²=0.6812) between the expectorated mouth-rinse liquid and the resting saliva can be employed as the conversion formula between the expectorated mouth-rinse liquid (x) and the resting saliva (y). In addition, it was found that a model y=0.3189x+27.477 (determination function R²=0.5326) between the resting saliva and the stimulated saliva can be employed as the conversion formula between the expectorated mouth-rinse liquid (x) and the stimulated saliva (y).

From the above, it can be appreciated that the conversion can be performed for at least one of the cariogenic bacteria, the occult blood, and the leukocytes having a correlation coefficient of 0.6 or more as a result of plotting the index measurement values, between the expectorated mouth-rinse liquid and the stimulated saliva. In addition, it can be appreciated that accuracy for the leukocytes having a correlation coefficient of 0.7 or more can be said to be high when converted. In the storage device 15 of the information processing device 10, a program capable of converting the measurement value (reflectance) of at least one of the cariogenic bacteria, the occult blood, and the leukocytes measured using one of the expectorated mouth-rinse liquid and the stimulated saliva into the converted value in the case of using the other of the expectorated mouth-rinse liquid and the stimulated saliva is installed.

### Measurement Using Inspection Device

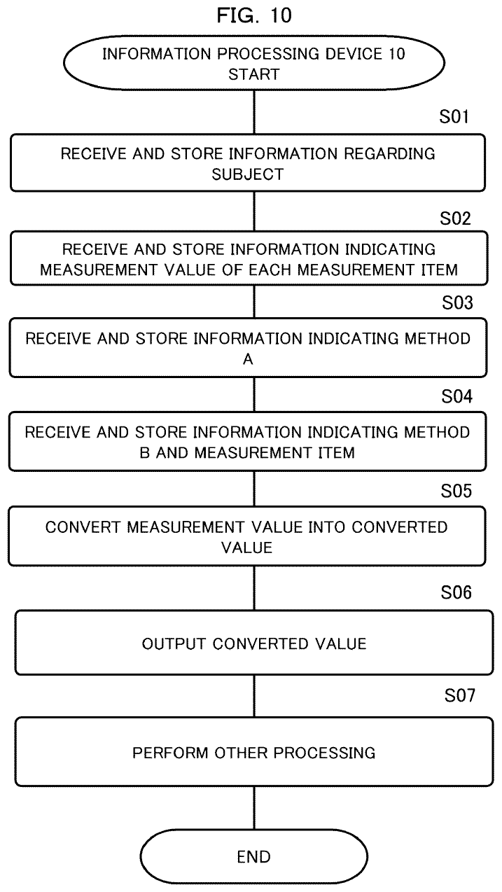

Next, a measurement method using the strip-shaped test paper 31 and the inspection device 30 will be described. FIG. 10 is a flowchart showing a processing example of the information processing device 10. The processing in each step in FIG. 10 is performed by the processor 11 which is the controller.

(1) Information regarding the subject (such as a patient) or the like is input to the information processing device 10 using the input device 12 and stored in the storage device 15 (S01 in FIG. 10).

(2) Saliva to be a specimen is collected from the subject using any one of a method for collecting the expectorated mouth-rinse liquid, a method for collecting the resting saliva, and a method for collecting the stimulated saliva.

(3) The specimen to be used for measurement is prepared according to the method for collecting saliva, the method being used for collection. Specifically, as in the above-described example, in a case where the collected specimen is the resting saliva or the stimulated saliva, the specimen is diluted 5 times with purified water.

(4) The specimen is spotted on the test pads 31a to 31g (FIG. 1) for the respective measurement items in the strip-shaped test paper 31.

(5) The strip-shaped test paper 31 after the spotting is installed in the measurement device 20.

(6) The measurement device 20 measures the predetermined measurement item (in the present example, the buffering capacity, the acidity, the cariogenic bacteria, the occult blood, the protein, the leukocytes, and the ammonia) and acquires each measurement value.

(7) A signal indicating each measurement value is transmitted from the measurement device 20 to the information processing device 10, and data indicating each measurement value is stored in the storage device 15 (S02 in FIG. 10).

(8) Using the input device 12 or the like, information indicating the saliva collection method (referred to as a method A) performed in the above (2) is input to the information processing device 10 (S03 of FIG. 10).

(9) Information designating a saliva collection method (referred to as method B) different from the method A and information designating a measurement item desired to be converted into a measurement value that can be obtained using the method B are input (S04 in FIG. 10). Note that the order of input of the method A, the method B, and the measurement method is arbitrary, and the method A, the method B, and the measurement method can be input at any timing in the processes (1) to (8).

(10) When a trigger for the conversion occurs in the information processing device 10 (for example, input of an instruction to perform the conversion, or a confirmation operation for the input of the method B and the measurement item), the processor 11 of the information processing device 10 performs processing of converting the measurement value according to the method A into the converted value according to the method B by executing the program (S05 in FIG. 10). As an example, the processor 11 calculates the converted value from the measurement value by performing calculation according to the calculation procedure for calculating the converted value from the measurement value of the measurement item.

(11) The converted value is output (S06 in FIG. 10). The converted value may be displayed on the display 14 or printed by the output device 40 under the control of the processor 11. In a case where there is a measurement value that has not been subjected to the conversion, the measurement value may be displayed or printed together with the converted value. In addition, the measurement value before the conversion may be displayed together with the converted value. In addition, the conversion formula may be displayed or printed together with the converted value.

(12) As other processing (S07 in FIG. 10), the processor of the information processing device 10 can perform determination regarding caries risk, periodontal disease risk, or the like for the periodontal disease stage by using the measurement value and the converted value, and can display a determination result for the periodontal disease stage on the display 14 or print the determination result by the output device 40. In this way, an inspection for determining the risk of oral disease or the level of oral disease is provided.

In the above-described example, an example has been described in which the information processing device 10 acquires the reflectance, which is the primary data of the measurement item measured by the measurement device 20, as the measurement value, and the processor 11 of the information processing device 10 converts the measurement value (reflectance) into the converted value (reflectance). However, the information processing device may acquire, from the measurement device 20 or the like, a value indicating the quantity of the measurement item calculated from the reflectance as the measurement value instead of the reflectance, and convert the measurement value into the converted value by using the conversion formula based on the value indicating the quantity.

With the information processing device according to the embodiment, for at least one measurement item selected from among the cariogenic bacteria, the occult blood, and the leukocytes among the seven measurement items described above, the measurement value obtained using the specimen (one of the expectorated mouth-rinse liquid and the stimulated saliva) collected by the method A can be converted into the converted value when collected by the method B (the other of the expectorated mouth-rinse liquid and the stimulated saliva). As a result, for example, in the case of performing a plurality of measurements using the expectorated mouth-rinse liquid, even if a certain number of measurements using the stimulated saliva are performed when a certain period of time for obtaining the expectorated mouth-rinse liquid cannot be secured, a value equivalent to that in the case of using the expectorated mouth-rinse liquid can be obtained by converting the measurement value into the converted value.

The same applies to a case where a plurality of measurements are performed using the stimulated saliva and one or more measurements are performed using the expectorated mouth-rinse liquid. In other words, even in a case where the measurement value obtained using the expectorated mouth-rinse liquid and the measurement value obtained using the stimulated saliva are mixed, the results can be treated as results obtained using either one of the expectorated mouth-rinse liquid and the stimulated saliva. That is, it is possible to estimate, from saliva collected using one collection method, the measurement value of saliva collected using the other collection method. As a result, as the inspection for determining the risk of oral disease or the level of oral disease, various determinations (determination regarding the caries risk and the periodontal disease risk) using the measurement results for saliva collected by different collection methods can be accurately performed. The configurations described in the embodiment can be appropriately combined.

All examples and conditional language recited herein are intended for pedagogical purposes to aid the reader in understanding the invention and the concepts contributed by the inventor to furthering the art, and are to be construed as being without limitation to such specifically recited examples and conditions, nor does the organization of such examples in the specification relate to a showing of the superiority and inferiority of the invention. Although the embodiments of the present invention have been described in detail, it should be understood that the various changes, substitutions, and alterations could be made hereto without departing from the spirit and scope of the invention.

What is claimed is:

1. A method for converting a measurement value related to saliva, the method comprising:
acquiring, from a measurement value of a measurement item using one of an expectorated mouth-rinse liquid and stimulated saliva collected from a subject, a converted value in a case of using another of the expectorated mouth-rinse liquid and the stimulated saliva collected from the subject, based on a correlation obtained in advance between an index measurement value of the measurement item measured in advance using the expectorated mouth-rinse liquid and an index measurement value of the measurement item measured in advance using the stimulated saliva, wherein
at least one of cariogenic bacteria, occult blood, and leukocytes is selected as the measurement item.

2. The method according to claim 1, wherein the measurement item is the leukocytes.

3. The method according to claim 1, wherein the measurement value is data indicating optical information of a test pad which has a reaction system corresponding to the measurement item and on which one of the expectorated mouth-rinse liquid and the stimulated saliva is spotted.

4. An information processing device comprising:
a controller configured to acquire, from a measurement value of a measurement item using one of an expectorated mouth-rinse liquid and stimulated saliva collected from a subject, a converted value in a case of using another of the expectorated mouth-rinse liquid and the stimulated saliva collected from the subject, based on a correlation obtained in advance between an index measurement value of the measurement item measured in advance using the expectorated mouth-rinse liquid and an index measurement value of the measurement item measured in advance using the stimulated saliva, and outputs the converted value,
wherein at least one of cariogenic bacteria, occult blood, and leukocytes is selected as the measurement item.

5. The information processing device according to claim 4, wherein the measurement value is data indicating optical information of a test pad which has a reaction system corresponding to the measurement item and on which one of the expectorated mouth-rinse liquid and the resting saliva is spotted.

6. The information processing device according to claim 4, wherein the controller is configured to acquire the converted value by executing a calculation procedure for obtaining the converted value from the measurement value, based on the correlation.

7. The information processing device according to claim 4, further comprising a storage configured to store information indicating the correlation, wherein the controller is configured to acquire the information indicating the correlation by reading the information indicating the correlation from the storage.

8. The information processing device according to claim 5, further comprising a communication interface is configured to receive information from an external device,
wherein the controller is configured to acquire information indicating the correlation by receiving the information indicating the correlation from the external device through the communication interface.

9. A non-transitory computer readable storage medium to store a program for causing a computer to execute:
acquiring, from a measurement value of a measurement item using one of an expectorated mouth-rinse liquid and stimulated saliva collected from a subject, a converted value in a case of using another of the expectorated mouth-rinse liquid and the stimulated saliva collected from the subject, based on a correlation between an index measurement value of the measurement item measured in advance using the expectorated mouth-rinse liquid and an index measurement value of the measurement item measured in advance using the stimulated saliva, and outputting the converted value,
wherein at least one of cariogenic bacteria, occult blood, and leukocytes is selected as the measurement item.
